(19) United States
(12) Patent Application Publication
(10) Pub. No.: US 2010/0041896 A1
(43) Pub. Date: Feb. 18, 2010

(54) Macrolide compound with bis-oxazoly

(76) Inventors: Zheng WEI, Fuzhou City (CN); Zhentai Cheng, Fuzhou City (CN); Tian-en Wei, Fuzhou City (CN); Yuanrong Cheng, Fuzhou City (CN); Yunyang Lian, Fuzhou City (CN); Wei Hung, Fuzhou City (CN)

Correspondence Address: Dr. BANGER SHIA;Int''l Patent Office of Bang Shia, 102 Lindencrest Ct, Sugar Land, TX, 77479-5201, US

(21) Appl. No.: 12/189,790

(22) Filed: Aug. 12, 2008

(51) Int. Cl. C07D 263/30

(52) U.S. Cl. 548/235

(57) ABSTRACT

This patent mainly provides a new macrolide compound named FW-04-806, which contains bis-oxazoly in molecule. FW-04-806, a bis-oxazolyl macrolide compound extracted and separated from the ferment liquid of streptomyces FIM-04-806, has anti-tumor activities and provides a chemical research basis for natural drugs and the lead compound for the research and development of new anti-tumor drugs. Anti-tumor in vitro experiment shows that minimal median inhibitory concentration IC₅₀ is 12.36 ug/ml to K562 cell (human chronic mydogenous leukemia leap clone).

### BACKGROUND OF THE INVENTION

(a) Field of the Invention

This invention relates to the technical field of biomedicine, which, specifically, is to get a new compound FW-04-806 with anti-tumor activity from the secondary metabolite of streptomyces FIM-04-806.

(b) Description of the Prior Art

Since penicillin was used in clinic in the beginning of 40s in the 19^{th} century, how to find bioactivity materials from microorganism had been the hotspot of medicament development. Since the first macrolide antibiotics—Erythromycin with clinic application worth was discovered by McGuire in 1952, macrolide antibiotics has been playing a rather important place in clinic. From perspective of action mechanism, these compounds act on the 50S ribosome subunit of the sensitive bacteria, and restrain protein synthesis of the bacteria through blocking spin-peptide activity and (or) mRNA dislocation, mainly used to restrain the growth of Gram-positive bacteria. But later on many new bioactivities of the macrolide compound were discovered successively, such as antivermin, antivirus, anti-tumor and enzyme inhibitor, etc.

In 1994, Jansen and others separated the bis-oxazoly compounds (disorazoles) containing 29 unique macro-ring polyketones from the zymotic fluid of polyangium strain (Sorangium Cellulosum Strain Soce12) (Jansen, R, et al. Liebigs Ann. Chem. 1994, 759-773.). All of which have centrally-symmetric dual large ring lacton and bis-oxazoly. It was discovered during the bioactivity test and effect mechanism exploration that these compounds may make the cell cycle stay in G2/M period and compete tubulin bind site with vincristine sulfate in vivo in order to restrain the polymerization activity of tubulin. The structural derivatives and physiological activity of this kind of compounds lead to the great attention from scientists and pharmacists. In 2002, the Germany Chan Fatalis Ltd. Co. applied the patent of using the compound and its derivatives as drugs for curing optimum and malignancy tumor diseases (Public No. CN 1678310A), and they discovered that this kind of bis-oxazoly compounds also have the cell toxic action to several human tumor cell series, which can restrain the cellular fission for ovary cancer, prostate cancer, colloid cell cancer, lung cancer and mammary cancer, etc. under Na mole and Pi mole concentration, and can be used as individual substance or in combination with other cell toxic substances, specially with the inhibitor of signal transduction.

### SUMMARY OF THE INVENTION

The main goal of this invention is to provide a new macrolide compound named FW-04-806 containing bis-oxazoly.

FW-04-806, a bis-oxazolyl macrolide compound extracted and separated from the ferment liquid of streptomyces FIM-04-806, has anti-tumor activities and provides a chemical research basis for natural drugs and the lead compound for the research and development of new anti-tumor drugs.

This invention provided a new compound FW-04-806 with anti-tumor activity of molecule formula (1), and provided a method extracting and refining the compound represented by molecule formula (1) obtained from the metabolite of the strain of streptomyces being cultivated.

The inventor of this patent discovered that a compound with anti-tumor activity existed in the secondary metabolite of streptomyces FIM-04-806 during process of searching new bioactivity substance from microorganism, and that the new compound FW-04-806 with molecule formula (1) has anti-tumor activity through separation, purification, and structural analysis of the compound. Anti-tumor in vitro experiment shows that minimal median inhibitory concentration IC₅₀ is 12.36 ug/ml to K562 cell (human chronic mydogenous leukemia leap clone).

1. The compound FW-04-806 has molecule formula as follows:

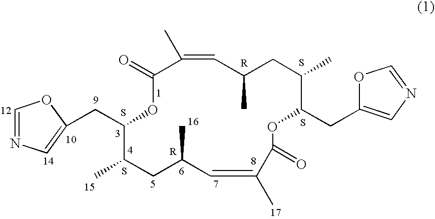

2. The method to extract and purify the compound FW04-806 from the culture.

The compound FW-04-806 represented by the molecule formula (1) above in this invention has following physical and chemical properties.

(1) Color and appearance: colorless or primrose yellow, crystal powder

(2) Molecular formula: C₂₈H₃₈N₂O₆

(3) Melting point: 120.5-122.5° C.

(4) Mass spectrum: (TOF-MS)
Measured value: m/z 499.2813 [M+H]⁺
Theoretical value: m/z 498.2808 [C₂₈H₃₈N₂O₆]⁺

(5) Ultraviolet absorption spectrum: λₘₐₓ, nm: 214.2/MeOH (see FIG. 1 attached)

(6) Infrared absorption spectrum: (KBr)
νₘₐₓ, cm⁻¹: 3133.29, 3095.11, 2962.97, 2874.42, 1698.11, 1603.07, 1509.85, 1274.95, 1096.72 (see FIG. 2 attached)

(7) ¹H nuclear magnetic resonance spectrum (CDCl₃, 500 MHz)
δ (ppm): 0.94(3H, d, J=7.1 Hz), 1.06(3H, d, J=6.6 Hz), 1.23(1H, mult), 1.40(1H, mult), 1.65(1H,mult), 1.70(3H, s), 2.53(1H, mult), 2.82(2H, Mult), 5.14(1H, mult), 6.28(1H, dd, J=10.40, 0.95 Hz), 6.74(1H, s), 7.73(1H, s) (see FIG. 3 attached)

(8) ¹³C nuclear magnetic resonance spectrum (CDCl₃, 500 MHz)
δ (ppm): 12.7, 16.6, 21.2, 24.5, 31.2, 35.3, 38.4, 74.1, 123.4, 127.5, 147.0, 149.3, 150.3, 166.3 (see FIG. 4 attached)

(9) Solubility: soluble in methyl alcohol, ethyl acetate and dimethylsulfoxide; nearly insoluble in n-hexane or petroleum ether; insoluble in water.

Firstly, culture Streptomyces FIM-04-806 which produces macrolide compound containing bis-oxazoly, and then ferment Streptomyces FIM-04-806. After centrifugation, discard the clear liquid of 10 ml zymotic fluid. Marinate the mycelium in alcohol for 12 hours. The volume of alcohol should be 5 times of the mycelium. After centrifugation, concentrate the clear alcohol solution at 60° C. under vacuum condition and concentrated alcohol extracting solution obtained; Add minimal volumes of methyl alcohol to the concentrated alcohol extracting solution. Add water 200 ml, ethyl acetate 200 ml for extraction. Obtain upper layer ethyl acetate extracting solution, and conduct vacuum concentration at 60° C. Add minimal volumes of methyl alcohol to solve the concentrated ethyl acetate extracting solution. Add water 200 ml and ether 200 ml. Obtain upper layer ether extracting solution. Concentrate ether extracting solution and then extract with 200 ml petroleum ether. The upper layer petroleum ether extracting solution is slowly separated out yellow crystal at 0° C. Purify it by a high performance counter current chromatographic instrument. Apply 254 nm ultraviolet for testing. Solution distribution system is shown as N-hexane:ethyl acetate:methyl alcohol:water=8:2:5:5. Conduct elution with upper phase being immobile phase and lower phase being mobile phase. After distillation, we obtain achromatism or primrose yellow crystal powder which is the macrolide compound containing bis-oxazoly described above. Its weight is about 2 g.

Described Streptomyces FIM-04-806 is cultured in shaking bottles. Gause's Agar is used as culture medium. The condition of shaking culture is described below. Temperature is between 25-30° C. Rotation speed is 220 rpm. Swing amplitude is 5 mm. Culture period is usually 6-10 days. PH value of the culture medium should be controlled between 7.0-8.0. The best culture temperature is 28° C.

The Components of seed culture medium: glucose 1% (w/v), amidulin 2% (w/v), soybean flour 1.5% (w/v), calcium carbonate 0.1% (w/v), PH 7.5, Put the components to triangular shaking bottles (500 ml) respectively, each 80 ml, then steamed and sterilized at 121° C. for 20 minutes, ready for use.

Inoculate FIM-04-806 grown in agar culture medium for 7 to 10 days to the shaking bottles (500 ml) each containing 80 ml seed culture medium, shake and culture them in a rotation bottle shaking machine (220 rpm) at 28° C. for about 48 to 70 hours.

Components of fermentation medium: starch 5% (w/v), barn powder 3% (w/v), peptone 1% (w/v), calcium carbonate 0.1%, PH 7.5, Put the components in tree shaking bottles(500 ml), each 80 ml, then, steamed and sterilized at 121° C. for 20 minutes, ready for use. Inoculate 1 to 3 ml seed culture fluid to the fermentation medium and shake and culture them in a rotation bottle shaking machine (220 rpm) at 28° C. for 5 to 7 days.

The advantage of the new bis-oxazoly macrolide compound in this invention is that it provides not only a new macrolide compound FW-04-806 containing bis-oxazoly, but also a method to extract and separate the compound FW-04-806 from the fermentation fluid of streptomyces FIM-04-806. The macrolide compound FW-04-806 containing bis-oxazoly has anti-tumor activity. This invention provides a chemical research basis of natural drugs and lead compounds for the research and development of new anti-tumor drugs.

### BRIEF DESCRIPTION OF THE DRAWINGS

The following is the further description of this invention referring to the attached Figs. according to the Implementation Cases.

FIG. 1 Ultraviolet absorption spectrum of FW-04-806.

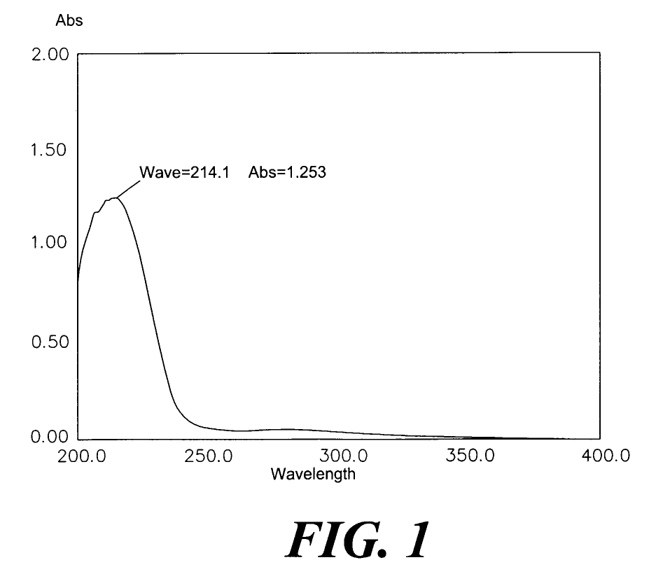

FIG. 2 Infrared absorption spectrum of FW-04-806.

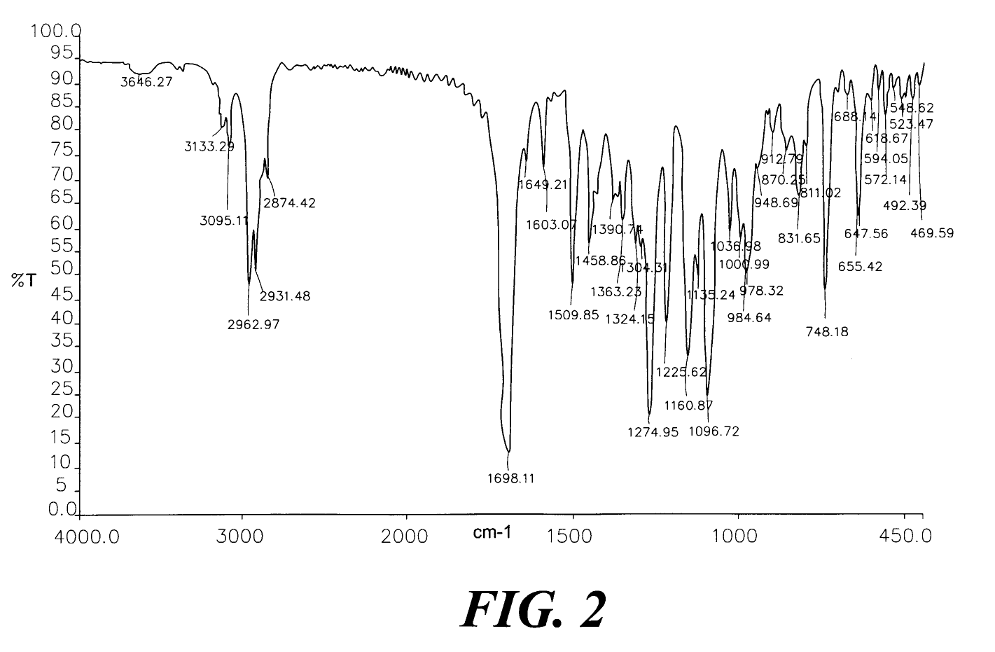

FIG. 3 ¹H nuclear magnetic resonance spectrum of FW-04-806.

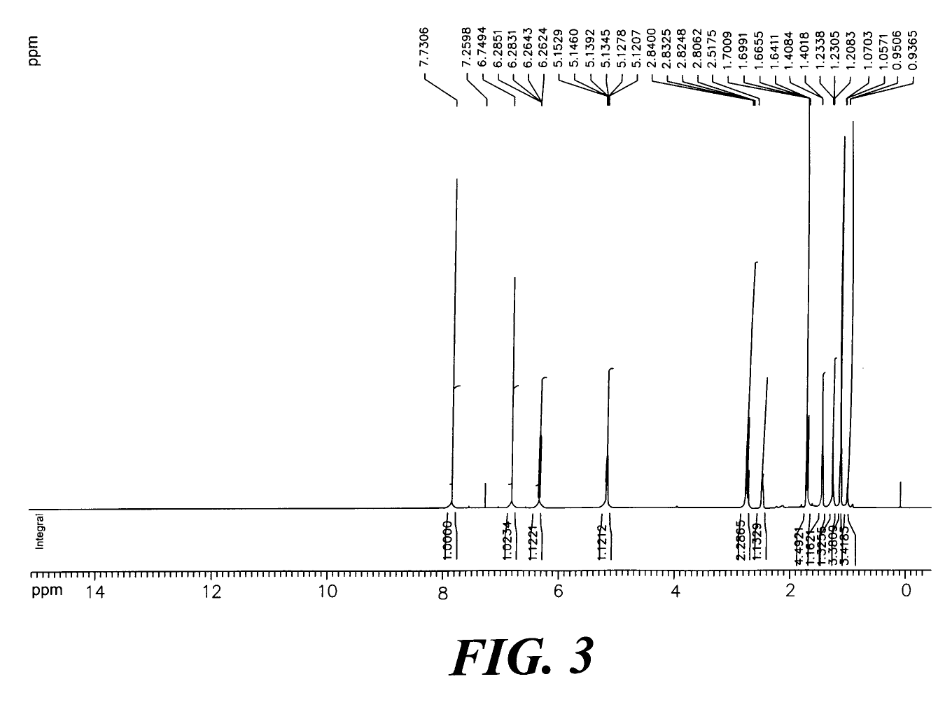

FIG. 4 ¹³C nuclear magnetic resonance spectrum of FW-04-806.

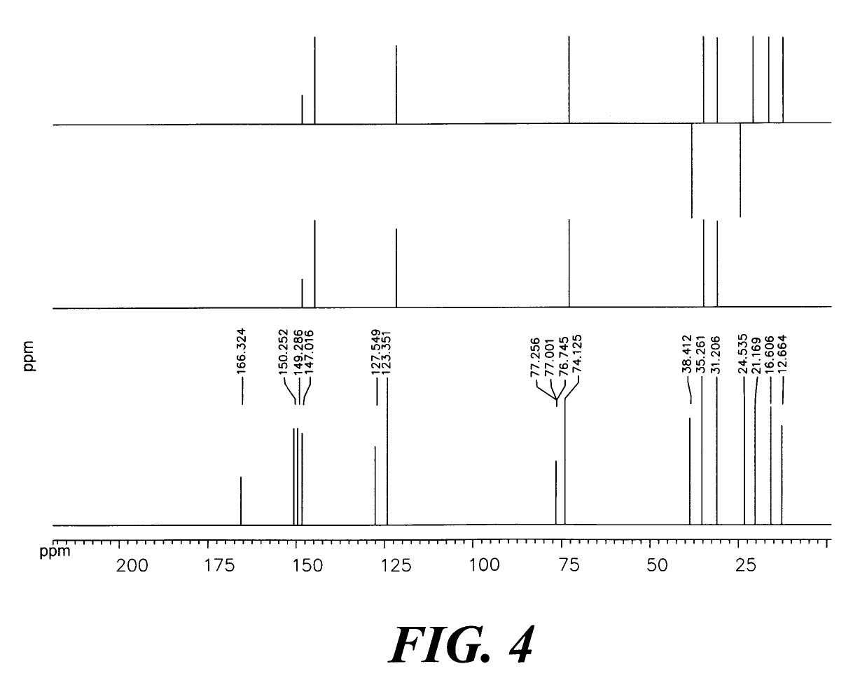

FIG. 5 Crystal structure spectrum of FW-04-806.

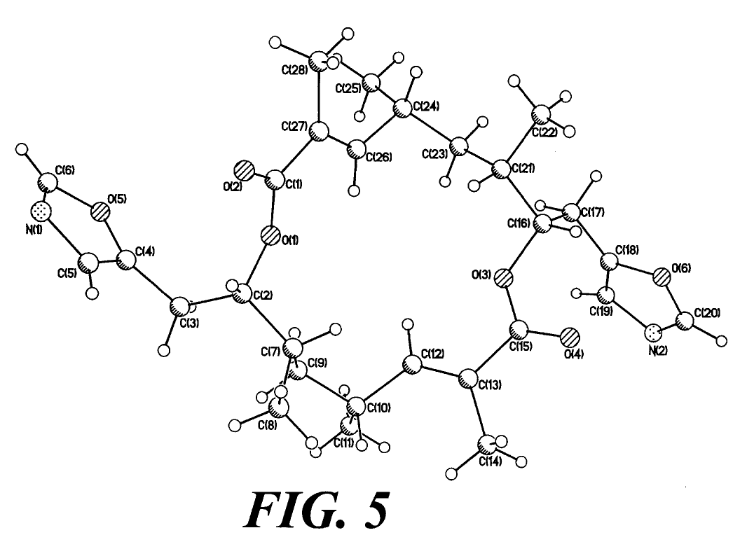

FIG. 6 Crystal X-ray diffraction data of FW-04-806.

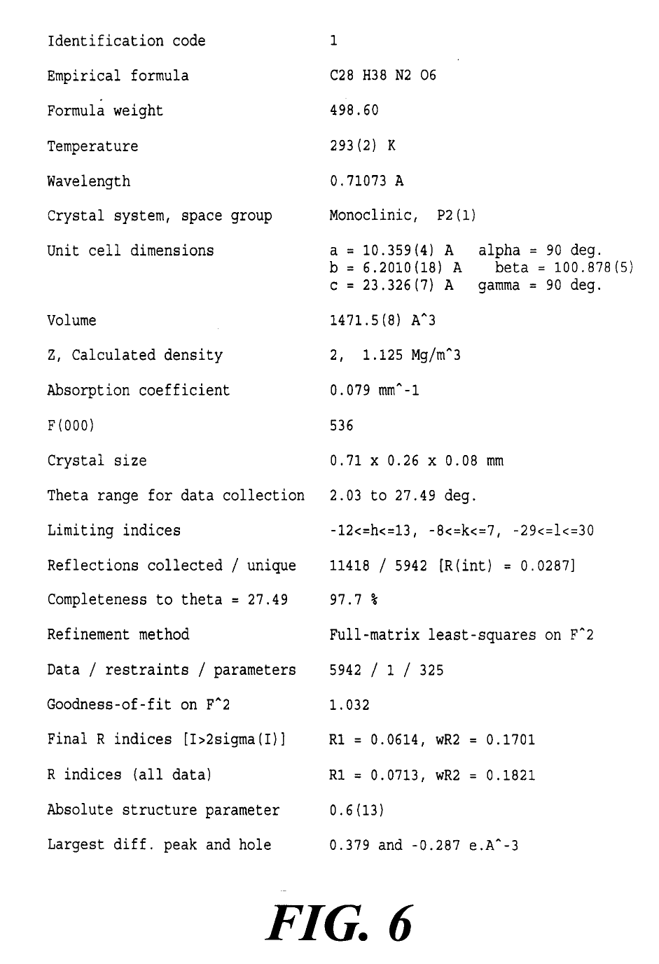

The bacterial microorganism strain of streptomyces FIM-04-806, which produces the compound FW-04-806, was already preserved in China General Microbiological Culture Collection Center on Jan. 18, 2008. Preservation number is CGMCC No. 2349.

### DETAILED DESCRIPTION OF THE PREFERRED EMBODIMENTS

The implementation Case 1: The fermentation conditions of Streptomyces FIM-04-806

The fungus FIM-04-806 which produces the compound FW-04-806 with molecular formula (1), grows in radiation on the agar growth medium, with no cross at mycelium, with loop, hook or short spiral-shaped sporothrix, and spore link including 10-30 spores. Whole-cell hydrolysate solution contains no characteristic sugar, a kind of sugar-C. Chemical composition of the cell wall is analyzed, containing L, L-2-G acid, aspartic acid, glycine, glutamic acid, alanine, belonging to the cell wall type I. The 16SrDNA sequence testing result of the strain has the highest homology with the strain of Streptomyces rimosus NBRC 12907 of the known sequences in GeneBank, reaching at 98.89 percent. According to the form of the strain FIM-04-806, the component analysis of the cell wall and 16SrDNA sequence testing result, it is shown that the strain is of Streptomyces.

The approach to produce compound FW-04-806 (molecular formula I) by fermentation in shaking flask:

Under 25-30° C. (optimum 28° C.) with 220 rpm rotation speed, the strain FIM-04-806 is oscillated and cultivated (swing amplitude 5 mm), producing a secondary metabolite FW-04-806 compound, usually in the culture cycle of 6-10 days. PH value of the culture medium is controlled between 7.0-8.0.

The composite of seed culture medium: the glucose 1% (w/v), soluble starch 2% (w/v), soybean powder 1.5% (w/v), calcium carbonate 0.1% (w/v), pH 7.5. Installed respectively in 500 ml triangular flask, 80 ml each, then steamed and sterilized for 20 minutes at 121° C., ready for use.

The FIM-04-806 taken from the agar growth medium having grown for 7-10 days is inoculated into 500 ml triangular shaking flask containing 80 ml seed culture medium. Under 28° C. with 220 rpm rotation speed, it is oscillated and cultivated for about 48-70 hours in the shaking flask.

Fermentation culture medium components: starch 5% (w/v), yeast powder 3% (w/v), peptone 1% (w/v), calcium carbonate 0.1% (w/v), pH value of 7.5. Installed respectively in 500 ml triangular shaking flask, 80 ml each, steamed and sterilized at 121° C. for 20 minutes for backup usage. Draw 1-3 ml seed culture medium liquid and put it into fermentation medium, under 28° C., with 220 rpm it is oscillated and cultivated for 5-7 days in the shaking flask.

The implementation case 2: FW-04-806 Preparation

The clear fluid is discarded from 10 L fermentation fluid after centrifugal, mycelium slag is soaked for 12 hours in alcohol of five times volume, and the clear ethanol fluid is concentrated at 60° C. in vacuum after centrifugal and ethanol extract concentrated liquid is obtained. Ethanol extract concentrated liquid is added the least volume of methanol (to be dissolvable is ok), 200 ml of water, and then 200 ml ethyl acetate extraction, obtaining the upper-layer ethyl acetate extract, and then concentrated at 60° C. in vacuum. Ethyl acetate concentrated liquid is dissolved with the least volume of methanol similarly, then adding 200 ml of water, and 200 ml ether, then the upper ether extract is obtained, which is concentrated, and then extracted using 200 ml petroleum ether. The upper-layer petroleum ether extract fluid is put under 0° C. and slowly precipitated yellow crystals. Then the efficient countercurrent chromatography (HSCCC-TBE300) is used for purification, and 254 nm UV is used for detection, with the solvent distribution system as n-hexane:ethyl acetate:methanol:water (8:2:5:5), and cleanse with the upper phase as stationary phase, and the lower phase as mobile phase. The solvent is steamed and dried to obtain about 2 g colorless or pale yellow crystalline powder which is the compound FW-04-806.

Implementation case 3: anti-tumor in vitro experiments

The cultivation condition for K562 cells (human chronic myeloid leukemia leap cell lines) is: the volume fraction of 10 percent of the calf serum RPMI1640, penicillin (100 IU/ml) and streptomycin (100 μg/ml), at 37° C., 5% CO₂ incubation box for cultivation. Take the logarithm growth period cells used for the inhibition rate experiments, with the concentration of cells inoculated 2.5×10⁵/ml. In the 96-pore cultivation board, K562 cells are vaccinated with the inoculation amount of about 5×10⁴/pore, 200 μl each pore. The drug is dissolved in dimethyl sulfoxide (DMSO), making the storage fluid of 10 mg/ml. Four groups are set up in this experiment, adding different concentrations of drugs respectively, so that the final concentrations are respectively 0.1, 1, 10, 100 μg/ml. There is no drug added for the reference control group, and three parallel holes are set up for each group, cultivating at 37° C. for 48 hours. Taking 0.4 percent trypan blue liquid (take 4 g trypan blue and put it into 100 ml PBS, making four percent trypan blue raw fluid, filtrated and disinfected, stored in 4° C. refrigerator, ready to use. Concentration is of 0.4 percent) and the cell suspension fluid, 50 μl respectively, mixing, and then take appropriate mixed fluid to fill in cell counting board, counting the four big grid non-coloring living cells.

Cell growth inhibition rate=(reference control group cells−cells in the experimental group)/control group cells×100%

The logarithms of different concentrations for the same drug are used to plot graph for the tumor cell growth inhibition rate, and the dose-response curve can be obtained, and the median inhibitory concentration IC₅₀ of the drug can be calculated according to the linear regression equation, that is, the drug concentration when the cell survival rate reduces 50 percent is 12.36 μg/ml.

Although the above described the specific implementation approach for the invention, the technical personnel familiar with this technical field should understand that what we described about the specific implementation cases is only illustrative and not used of limiting the scope of the technical line for the invention, and the compound entity obtained from any kind of microbial cultures and the derivatives modified or converted from the compound entity through chemical or micro-organisms should be covered within the scope of protection in the claims of the invention.

I claim:

1. A macrolide compound containing bis-oxazoly, with its characteristics as: its molecular formula is:

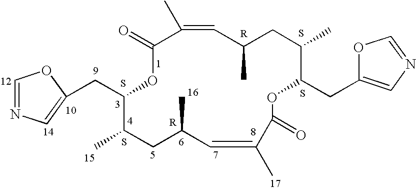

2. As in above-stated claim 1, a macrolide compound containing bis-oxazoly, with its characteristics as: its physical and chemical properties as the following:
(1) color and appearance: colorless or pale yellow crystalline powder
(2) molecular formula C₃₈H₃₈N₂O₆
(3) the melting point: 120.5-122.5° C.
(4) MS: (TOF-MS)
Measurement: m/z 499.2813 [M+H]⁺
The theoretical value: m/z 498.2808 [C28H38N2O6]⁺
(5) UV absorption spectra: λₘₐₓ, nm: 214.2/MeOH
(6) infrared absorption spectrum: (KBr)
νₘₐₓ, cm−1: 3133.29, 3095.11, 2962.97, 2874.42, 1698.11, 1603.07, 1509.85, 1274.95, 1096.72
(7) ¹H magnetic resonance spectroscopy (CDCl₃, 500 MHz)
δ (ppm): 0.94 (3H, d, J=7.1 Hz), 1.06 (3H, d, J=6.6 Hz), 1.23 (1H, mult), 1.40 (1H, mult), 1.65 (1H, mult), 1.70 (3H, s), 2.53 (1H, mult), 2.82 (2H, Mult), 5.14 (1H, mult), 6.28 (1H, dd, J=10.40, 0.95 Hz), 6.74 (1H, s), 7.73 (1H, s)
(8) ¹³C nuclear magnetic resonance spectroscopy (CDCl₃, 500 MHz)
δ (ppm): 12.7, 16.6, 21.2, 24.5, 31.2, 35.3, 38.4, 74.1, 123.4, 127.5, 147.0, 149.3, 150.3, 166.3
(9) solubility: soluble in methanol, ethyl acetate and DMSO; slightly soluble n-hexane and petroleum ether; do not dissolve in water.

3. As in above-stated claim 1, a macrolide compound containing bis-oxazoly, with its characteristics as: its production method is: Firstly, culture Streptomyces FIM-04-806 which produces macrolide compound containing bis-oxazoly, and then ferment to Streptomyces FIM-04-806. The clear fluid is discarded from 10 L fermentation fluid after centrifugal, mycelium slag is soaked for 12 hours in alcohol of five times volume, and the clear ethanol fluid is concentrated at 60° C. in vacuum after centrifugal and ethanol extract concentrated liquid is obtained. Ethanol extract concentrated liquid is added the least volume of methanol (to be dissolvable is ok), 200 ml of water, and then 200 ml ethyl acetate extraction, obtaining the upper-layer ethyl acetate extract, and then concentrated at 60° C. in vacuum. Ethyl acetate concentrated liquid is dissolved with the least volume of methanol similarly, then adding 200 ml of water, and 200 ml ether, then the upper ether extract is obtained, which is concentrated, and then extracted using 200 ml petroleum ether. The upper-layer petroleum ether extract fluid is put under 0° C. and slowly precipitated yellow crystals. Then the efficient countercurrent chromatography (HSCCC-TBE300) is used for purification, and 254 nm UV is used for detection, with the solvent distribution system as n-hexane:ethyl acetate:methanol:water (8:2:5:5), and cleanse with the upper phase as stationary phase, and the lower phase as mobile phase. The solvent is steamed and dried to obtain about 2 g colorless or pale yellow crystalline powder which is the compound FW-04-806.

4. As in above-stated claim 3, a macrolide compound containing bis-oxazoly, with its characteristics as: The above-stated Streptomyces FIM-04-806 is cultivated in the shaking flask, using agar culture medium, under 25-30° C., with 220 rpm rotation speed, oscillated and cultivated with swing amplitude of 5 mm, usually in the cultivation cycle of 6-10 days. PH value of the medium is controlled between 7.0 to 8.0.

5. As in above-stated claim 4, a macrolide compound containing bis-oxazoly, with its characteristics as:
The composite of seed medium: the glucose 1% (w/v), soluble starch 2% (w/v), soybean powder 1.5% (w/v), calcium carbonate 0.1% (w/v), pH 7.5. Installed respectively in 500 ml triangular shaking flask, 80 ml each, then steamed and sterilized for 20 minutes at 121° C., ready for use.
The FIM-04-806 taken from the agar growth medium having grown for 7-10 days is inoculated to 500 ml triangular shaking flask containing 80 ml seed culture medium. Under 28° C., with 220 rpm rotation speed, it is oscillated and cultivated for about 48-70 hours in the shaking flask.
Fermentation culture medium components: starch 5% (w/v), yeast powder 3% (w/v), peptone 1% (w/v), calcium carbonate 0.1% (w/v), pH value of 7.5. Installed respectively in 500 ml triangular shaking flask, 80 ml each, steamed and sterilized at 121° C. for 20 minutes, ready for use. Draw 1-3 ml seed culture medium liquid and put it into fermentation medium, under 28° C., with 220 rpm it is oscillated and cultivated for 5-7 days in the shaking flask.

6. As in above-stated claim 4, a macrolide compound containing bis-oxazoly, with its characteristics as: the culture temperature is 28° C.
